(19) United States
(12) Patent Application Publication
(10) Pub. No.: US 2015/0290016 A1
(43) Pub. Date: Oct. 15, 2015

(54) ORTHOPEDIC DEVICE

(71) Applicant: A STEP FORWARD, LLC, New York, NY (US)

(72) Inventor: Charles Sommer, New York, NY (US)

(21) Appl. No.: 14/685,296

(22) Filed: Apr. 13, 2015

Related U.S. Application Data
(60) Provisional application No. 61/979,569, filed on Apr. 15, 2014.

(51) Int. Cl. A61F 5/01

(52) U.S. Cl. CPC A61F 5/0195; A61F 2005/0197

(57) ABSTRACT

An orthopedic device is presented which comprise a horizontal base and a vertical support. The vertical support is connected toward the rear end of the horizontal base by a pivot mechanism and a tension mechanism. The pivot mechanism may include slots which allow the position of the vertical support to be adjusted. The tension mechanism may be a band such as an o-ring which is connected to both a first protrusion on the vertical support and a second protrusion on the rear end of the horizontal base. A locking mechanism may also be present to further connect the vertical support to the horizontal base.

This application claims the benefit of the filing date of U.S. Provisional App. No. 61/979,569, filed Apr. 15, 2014, which is hereby incorporated by reference in its entirety.

### FIELD OF THE INVENTION

The invention relates to an orthopedic device. More specifically, the invention relates to an orthopedic footwear with a pivot mechanism and a tension mechanism.

### BACKGROUND

Traditionally, after a patient receives surgery on the foot area (e.g., toes, ankle, Achilles, heel, etc.), the patient is required to wear an orthopedic device on the foot to stabilize the foot area. The orthopedic device also helps to relieve pressure on the foot area as well as helps to minimize sudden or excessive movements which may damage the foot area. The orthopedic device is typically in the form of an orthopedic footwear which has a horizontal base connected at the rear end to a rigid vertical support. The orthopedic footwear is secured to the patient's foot by means of a strap or other well known attachment means.

However, due to the rigid or inflexible vertical support, conventional orthopedic footwears are often uncomfortable to wear for extended periods of time. More specifically, because the rear vertical support is rigid, it does not bend in unison with the natural movement of a patient's leg when walking. As a result, the rigid rear vertical support puts unnecessary restriction and strain on the patient's lower leg, which can hamper its recovery. Patients often find conventional orthopedic footwear to be cumbersome and impede the natural walking movement of their leg.

As such, there is a need for a new orthopedic device Which will better adjust to the patient's natural leg movements so that the patient will feel more comfortable and recovery will improve.

### SUMMARY

A new and novel orthopedic device has been invented to overcome the unnecessary problems of conventional orthopedic devices. The orthopedic device comprise a horizontal base and a vertical support, wherein the vertical support is connected toward the rear end of said horizontal base by a pivot mechanism and a tension mechanism. The pivot mechanism can include slots to adjust the location of the pivot axle on the horizontal base. The tension mechanism may include bands such as o-rings which attach/connect to a first protrusion on the vertical support and a second protrusion on the horizontal base. A locking mechanism may also be present to further connect the rear end of the horizontal base to the vertical support.

Furthermore, a new and novel method for treating foot or leg surgery has also been discovered using the orthopedic device of the present invention.

### BRIEF DESCRIPTION OF THE DRAWINGS

FIG. 1 is an illustration of an orthopedic device of the present invention.

FIG. 2 is an illustration of the rear view of an orthopedic device of the present invention.

FIG. 3 is an illustration of the top view an orthopedic device of the present invention.

FIG. 4 is an illustration of an orthopedic device of the present invention with the patient leg having a forward motion.

FIG. 5 is an illustration of an orthopedic device of the present invention with the patient leg in the middle position.

FIG. 6 is an illustration of an orthopedic device of the present invention with the patient leg having a backward motion.

FIG. 7 is an illustration of an orthopedic device of the present invention.

FIG. 8 is an illustration of the rear view of an orthopedic device of the present invention.

FIG. 9 is an illustration of the top view an orthopedic device of the present invention.

FIG. 10 is an illustration of the rear perspective review of an orthopedic device of the present invention.

FIG. 11 is an illustration of an exploded perspective view of an orthopedic device of the present invention.

FIG. 12 is an illustration of a perspective view of an orthopedic device of the present invention.

FIG. 13 is an illustration of an exploded perspective view of an orthopedic device of the present invention.

FIG. 14 is an illustration of an orthopedic device of the present invention.

FIG. 15 is an illustration of the rear view of an orthopedic device of the present invention.

FIG. 16 is an illustration of the top view an orthopedic device of the present invention.

FIG. 17 is an illustration of an orthopedic device of the present invention in the forward position.

FIG. 18 is an illustration of an orthopedic device of the present invention in the middle position.

FIG. 19 is an illustration of an orthopedic device of the present invention in the backward position.

FIG. 20 is an illustration of a perspective view of an orthopedic device of the present invention

FIG. 21 is an illustration of an exploded perspective view of an orthopedic device of the present invention.

FIG. 22 is an illustration of an orthopedic device of the present invention.

FIG. 23 is an illustration of the rear view of an orthopedic device of the present invention.

FIG. 24 is an illustration of the top view an orthopedic device of the present invention.

### DETAILED DESCRIPTION

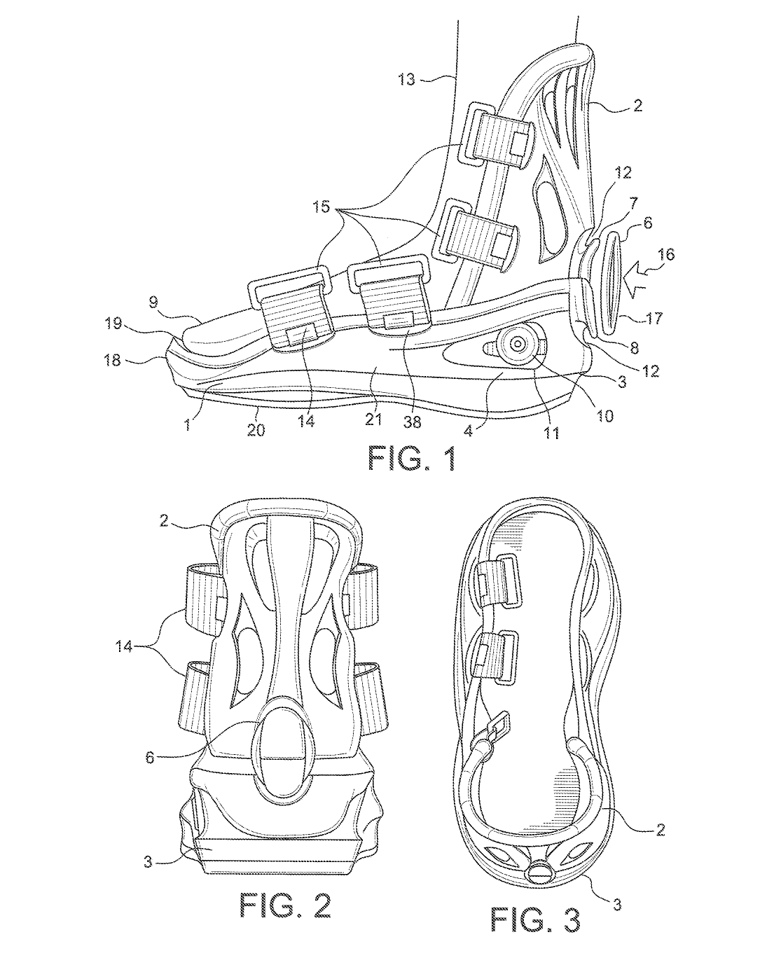

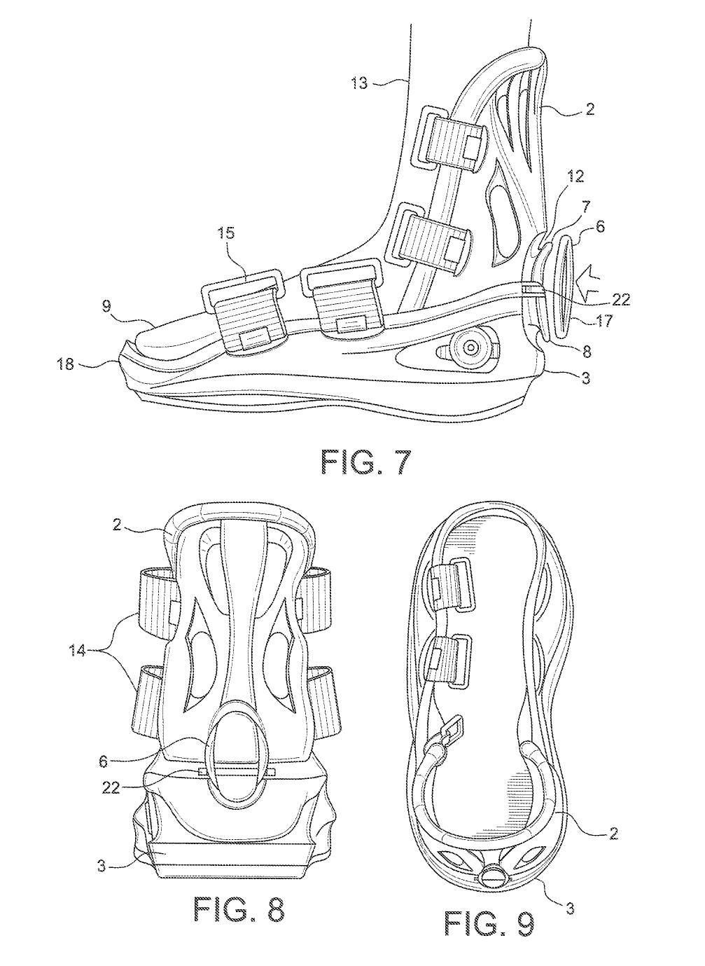

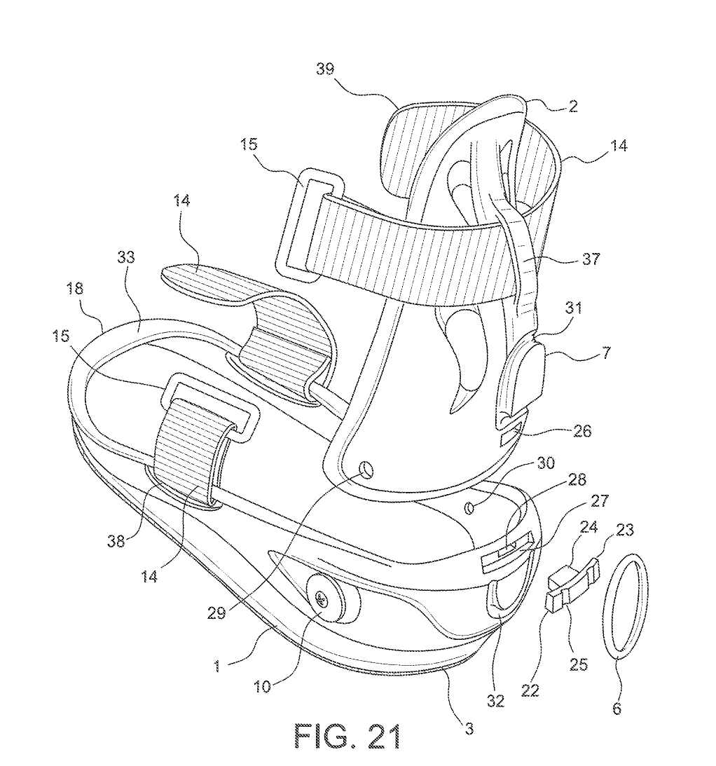

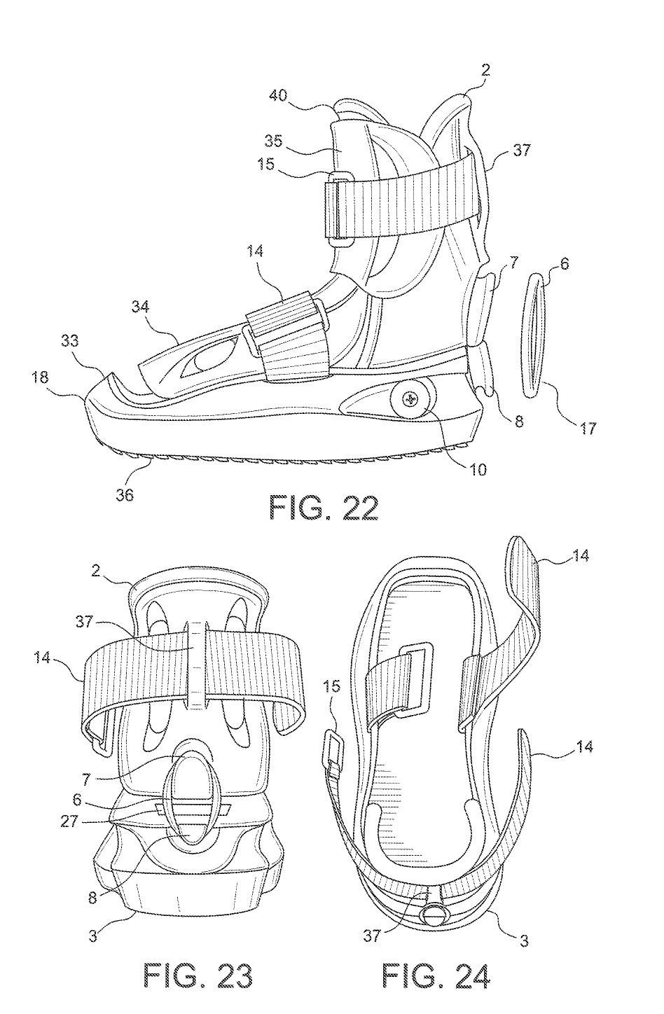

As shown in FIGS. 1-24, the orthopedic device of the present invention comprises a horizontal base 1 and a vertical support 2. The vertical support 2 is connected toward the rear end 3 of the horizontal base 1 by a pivot mechanism (or means) 4 and a tension mechanism (or means) 16. The pivot mechanism 4 may include slots 5 which allow the pivot axle 10 (and thus the position of vertical support 2) to be adjusted forward or backward along the length of the horizontal base 1. The tension mechanism 16 may be a band 17 such as an o-ring 6 which is connected to both a first protrusion 7 on the vertical support 2 and a second protrusion 8 on the rear end 3 of the horizontal base 1. A locking mechanism (or s) 22 may also be present to further connect the vertical support 2 to the horizontal base 1.

A fastener or attachment mechanism (or means) 39 such as a combination of straps 14 and buckles 15 can also be provided to help keep the orthopedic device on the patient.

Horizontal Base

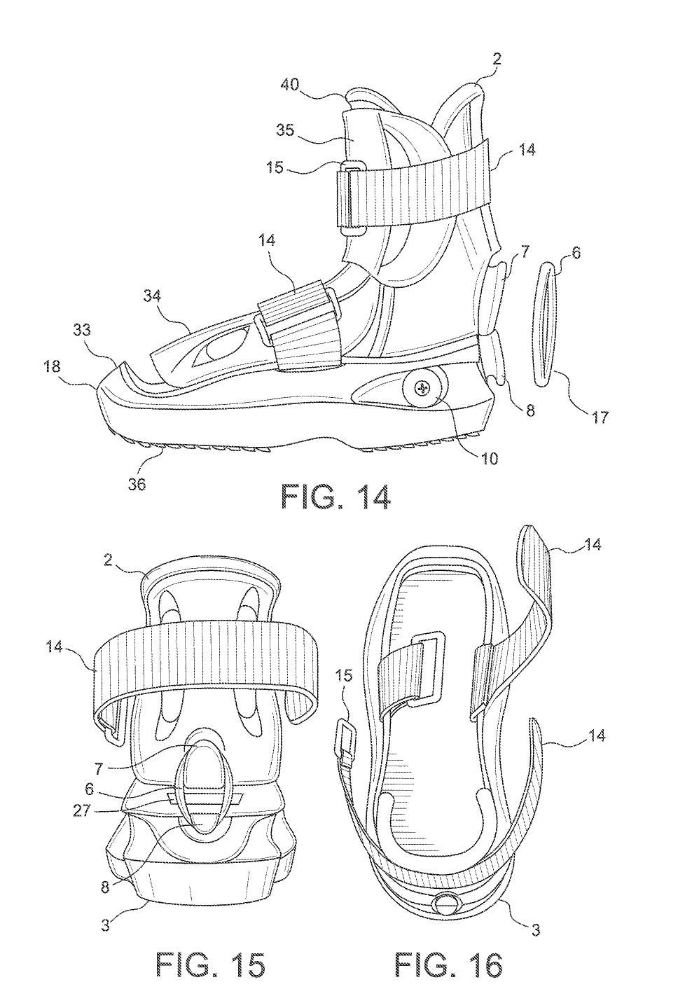

The horizontal base 1 provides a support for the foot 9 to rest. The toes of foot 9 would rest at the front end 18 of the horizontal base 1. The heel of the foot 9 would rest at the rear end 3 of the horizontal base 1. The horizontal base 1 has a top surface 19 and bottom surface 20, as well as side surface 21 on each side of the base. As shown in FIGS. 14, 18-19 and 22, the bottom surface 20 can include ridges 36 to better grip or cushion against the walking surface.

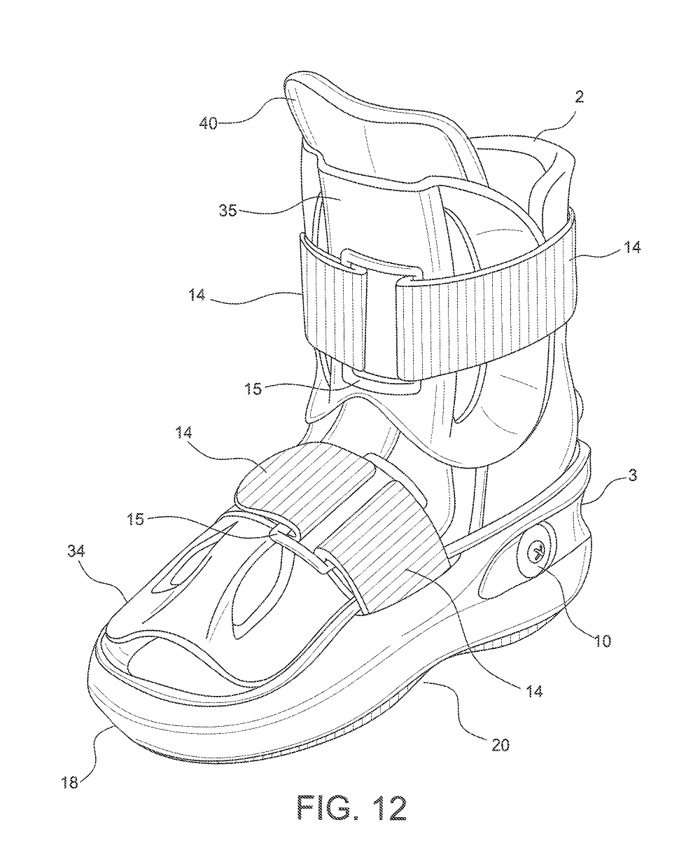

The horizontal base 1 can be custom molded to fit the shape of the patient's foot 9, or can be any standard shape conventionally used in the art for a patient's foot. For example, the horizontal base 1 can take on a flatter and more elliptical shape (e.g., compare FIG. 20-22 versus FIG. 12-14) to provide more support for the foot if needed. The horizontal base 1 can be of any thickness desired (e.g., from 1-6 inches thick, 2-5 inches thick, 3-4 inches thick, etc.). The horizontal base 1 can also be tapered in thickness (e.g., having a thinner thickness toward the front of the foot and a thicker thickness toward the rear of the foot).

To further protect the toes from harm, the horizontal base 1 can also have a lip 33 (e.g., a protective cover, shield or shelter) at the front end 18 that extends inward at an angle to cover the patient's toes.

The horizontal base 1 may also include openings 38 for connecting or securing any attachment mechanism or means thereto.

The horizontal base 1 can be made of any materials conventionally known or used in orthopedic devices, including but not limited to polymers, plastic, foam, rubber, etc. The horizontal base 1 can be made using any conventional process known in the art, and can also be made using a 3-D printer.

Vertical Support

The vertical support 2 is connected to or toward the rear end 3 of the horizontal base 1. The vertical support 2 is designed to provide support for the ankle, achilles and/or rear area of the lower leg. The vertical support 2 is preferably comprised of a single support piece having a curved/concave/semi-circle configuration such as a cuff to fit the shape of the rear area of the lower leg. Preferably, the vertical support 2 has a diameter that is larger than the diameter of the patient's lower leg/ankle area. Alternatively, the vertical support 2 has a circumference that is larger than the circumference of the patient's lower leg/ankle area.

The vertical support 2 may also include openings 37 for connecting or securing any fastener or attachment mechanism or means thereto.

The vertical support 2 can be made of any materials conventionally known or used in orthopedic devices, including but not limited to polymers, plastic, foam, rubber, etc. The vertical support 2 can be made using any conventional process known in the art, and can also be made using a 3-D printer. The vertical support 2 can be made of the same or different material as the horizontal base 1.

Pivot Mechanism

The pivot mechanism (or means) 4 connects the vertical support 2 to the horizontal base 1. Preferably, the pivot mechanism 4 is located toward the rear of the horizontal base 1.

The pivot mechanism 4 may comprise an axle (or rod) 10 by which the vertical support 2 pivots forward or backward with the motion of the lower leg 13. There is preferably an axle 10 on each side of horizontal base 1.

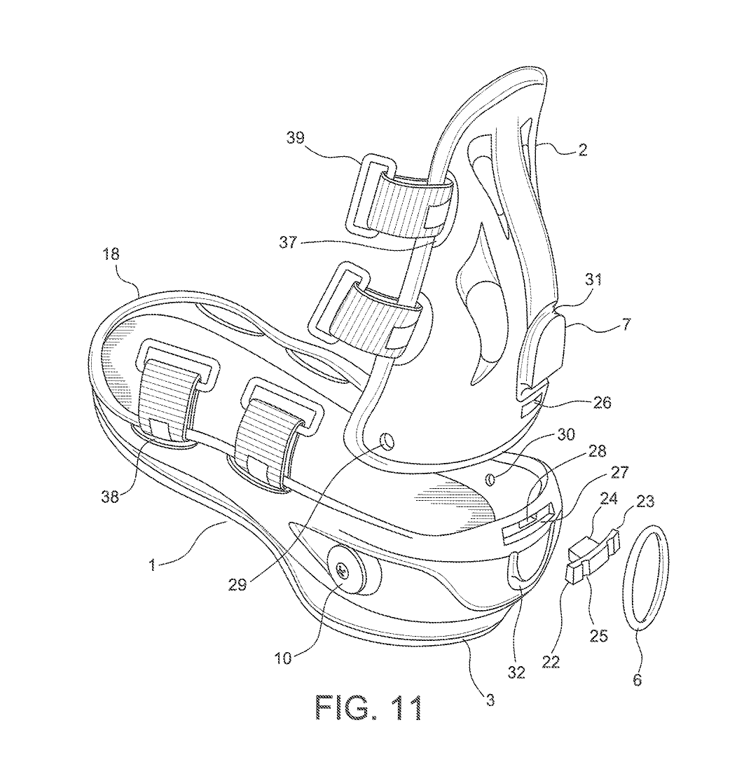

As shown in FIGS. 11, 13 and 21, the axle 10 is inserted into a first pivot hole 30 on horizontal support 1 and the corresponding second pivot hole 29 on the vertical support 2. A pivot hole is simply any hole or opening which receives the axle 10.

The axle 10 can include threads on the axle itself for screwing into the holes 30 and 29, or by which a nut can be screwed on to tighten and hold the vertical support 2 into place once attached to horizontal base 1. Alternatively, the axle 10 can be configured using any conventional male/female connector arrangement known in the art to secure the vertical support 2 to the horizontal base 1.

Additionally, washers can be placed on the axle 10 in between the horizontal base 1 and the vertical support 2 to prevent them from grinding or wearing down. These washers can be lubricated or made of nonabrasive smooth material such as nylon or polypropylene that will allow the vertical support 2 to rotate or pivot with the lower leg 13 along axle 10 with less friction than a direct contact with the walls of horizontal base 1.

There may be a recess 11 by which the axle 10 resides in toward the rear of the horizontal base 1. Additionally, the recess 11 may include a plurality of slots 5 for the axle 10 to rest upon. The plurality of slots 5 allow the position of axle 10 (and thus vertical support 2) to be adjusted forward or backward along the length of the horizontal base 1 in order to accommodate different foot sizes.

Tension Mechanism

The tension mechanism (or means) 16 also connects the rear end 3 of horizontal base 1 to the vertical support 2. The purpose of the tension mechanism 16 is to provide additional resistance and/or support for the rear of the patient's lower leg 13.

The tension mechanism 16 may be a band 17 such as an o-ring 6 which is connected to both a first protrusion 7 on the vertical support 2 and a second protrusion 8 on the rear end 3 of the horizontal base 1. To better improve the band's connection to the protrusions and 8, there may be a depression (or recess, indentation, etc) 12 toward the interior of the protrusions 7 and 8 by which the band 17 such as the o-ring 6 can fit snugly into. More specifically, the depression 12 may comprise a first depression 31 on vertical support 2 and a second depression 32 on horizontal base 1. The depression 12 can be designed to be of the same or similar shape to the band 17 such as the shape of o-ring 6.

An o-ring is a well known term of art referring to a closed loop connector ring which is typically in a shape of a circle or an oval. However, one skilled in the art will understand that o-ring 6 or band 17 can take any shape known in the art (square, rectangle, triangle, irregular shape, etc.) The band 17 or o-ring 6 can be made from rubber, polymer, composites thereof, or any other conventional substance known in the art for forming bands or o-rings.

The tension of the tension mechanism can be adjusted by using different bands or having different shape, diameter, thickness, elasticity or stiffness. The elasticity or stiffness of the band will be based on the material used to form the band. One commonly used measure of stiffness is the Young's modulus, which is also known as the tensile modulus or elastic modulus. The higher the Young's modulus, the more rigid and higher the tension of the connector band or o-ring. The lower the Young's modulus, the less rigid and lower the tension of the band. For example, the Young's modulus of the band can vary between 0.01-5.00 GPa depending on the material used and tension desired (e.g., 0.01-0.1 GPa for rubber, 2-4 GPa for nylon, etc.) Other Young's modulus range can be between 0.1-3.0 GPa, or 0.1-2.0 GPa, or 0.1-1.0 GPa, or 0.1-0.5 GPa). Other elastic moduli measurements include bult modulus and the shear modulus.

As such, a plurality of bands or o-rings can be provided so that the patient or doctor can adjust the tension or stiffness of the tension mechanism as needed. So that the patient or doctor will know how tense the band is, a tension indicator (or value) can be labeled, printed or marked onto the band itself. The tension indicator can be for example an assigned number or letter range representing a sliding scale of tension (e.g., 1 represents a lower tension value, 5 represents a high tension value, etc.) Or the tension indicator can simply be the actual tension property (e.g., Young's modulus value) of the band.

Alternatively, the tension indicator can be a color (i.e., band can be-color coded to indicate the tension value) (e.g., green o-ring represents a lower tension/stiffness, red o-ring represents a higher tension/stiffness, etc.)

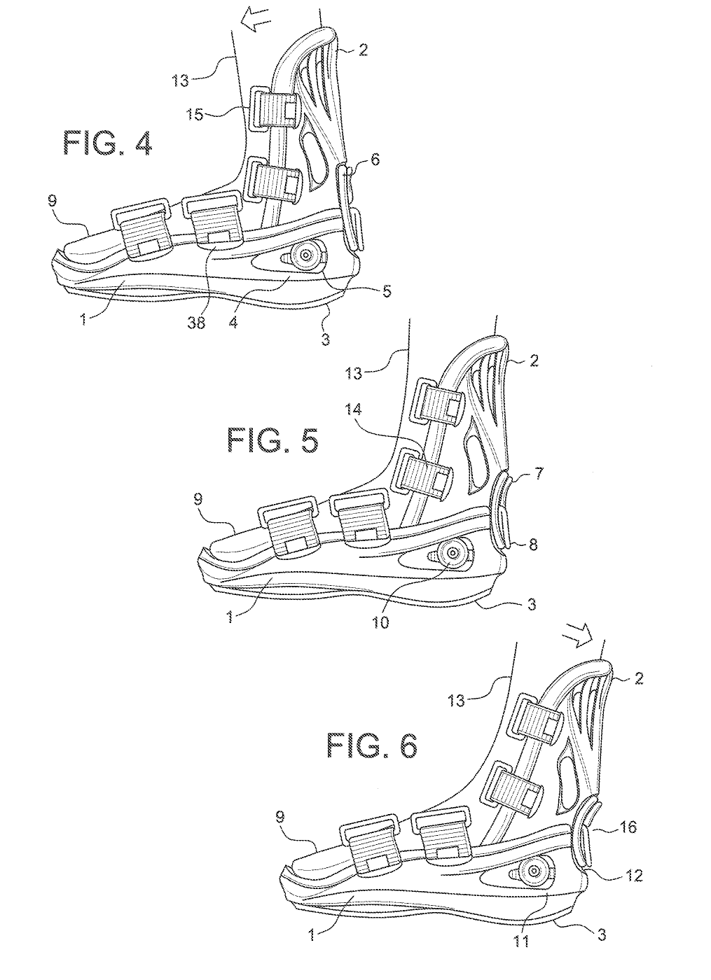

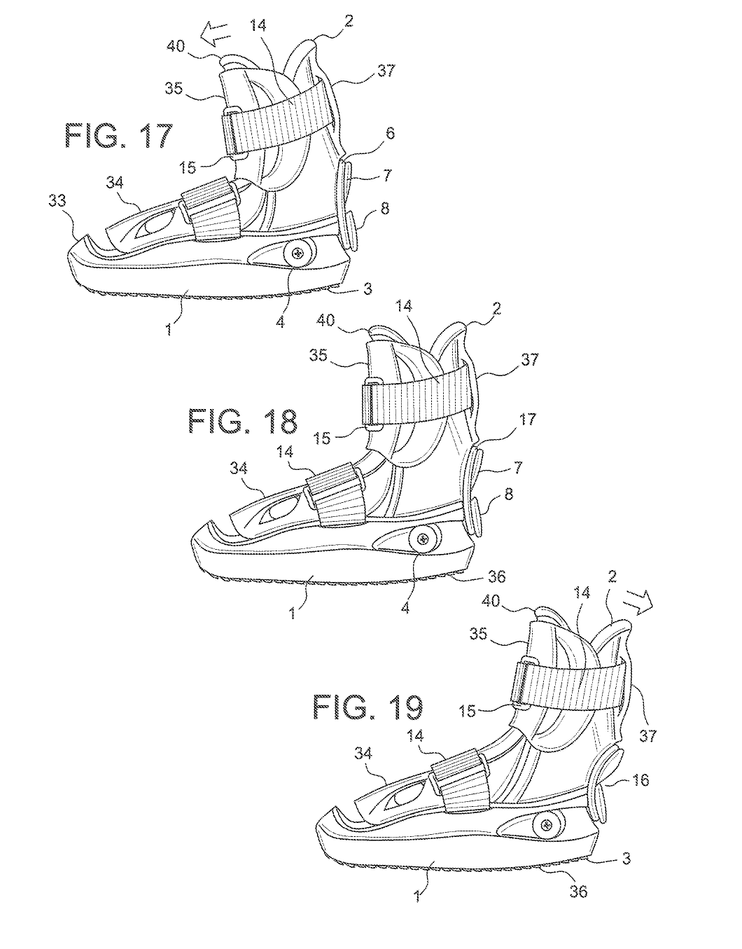

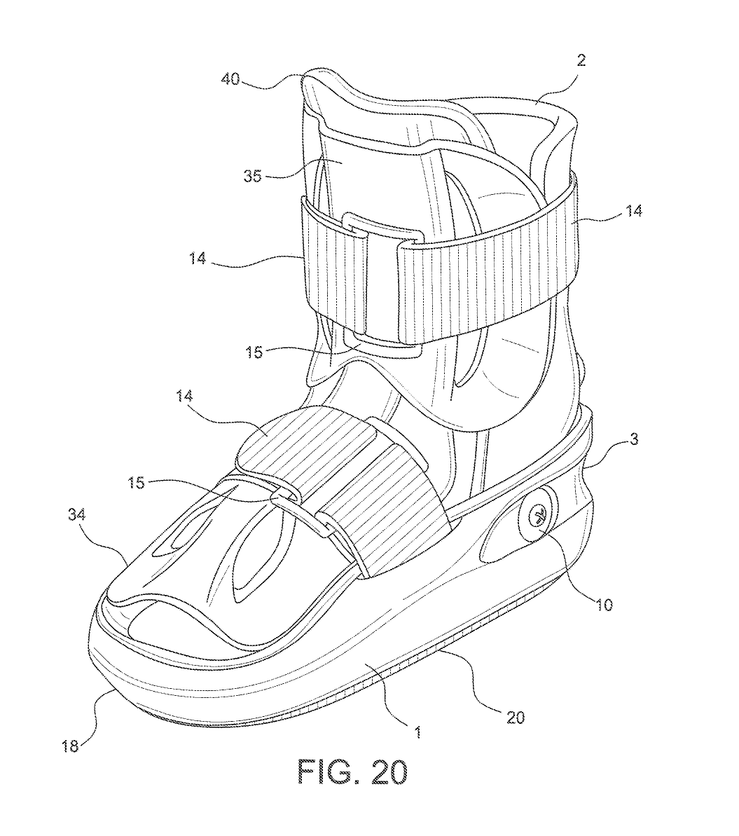

FIGS. 4-6 and 17-19 show how the vertical support 2 bends with the natural movement of the patient's lower leg 13 during walking motion. In FIGS. 4 and 17, then the patient walks forward, the lower leg 13 (shown in FIG. 4) moves forward as indicated by the arrow. The vertical support 2 also bends forward with the lower leg 13. In FIGS. 5 and 18, when the lower leg 13 (shown in FIG. 5) returns to a middle position, the vertical support 2 follows the lower leg 13 back to the middle position. In FIGS. 6 and 19, when the lower leg 13 (shown in FIG. 6) moves backward as indicated by the arrow, the vertical support 2 pivots backward with the lower leg 13. However, due to the tension provided by the band 17 such as an o-ring 6, the vertical support 2 is biased to return to its middle position (FIGS. 5 and 18) and thus provides some resistance to bending/pivoting backward, thereby providing additional support for the lower leg 13.

Locking Mechanism

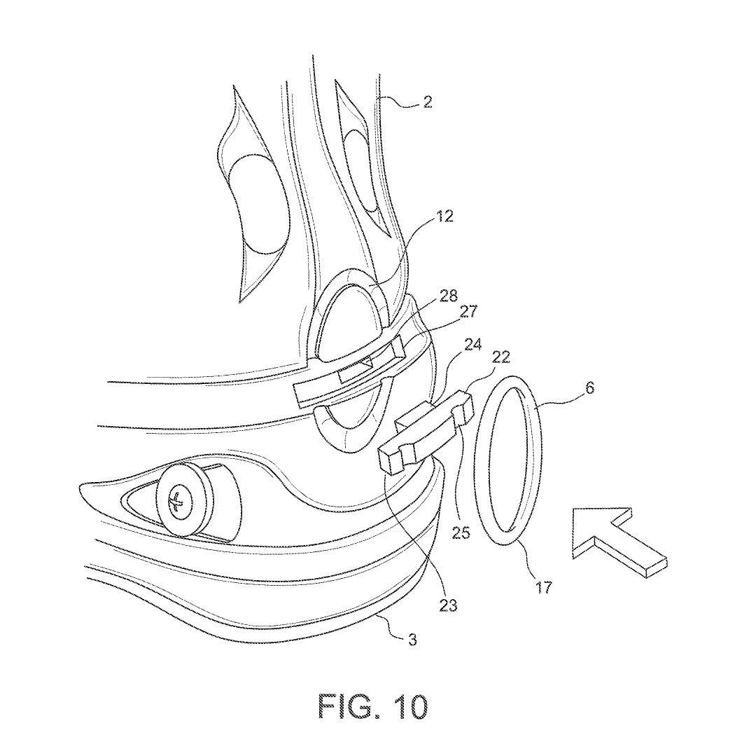

There may also be a locking mechanism (or means) 22 to further hold the rear end 3 of horizontal base 1 to the vertical support 2. As shown in FIGS. 10, 11, 13 and 21, the locking mechanism 21 may include an outer portion 23 having a first width and an inner portion 24 having a narrower second width. The locking mechanism 22 may mate with corresponding locking hole 27 of the horizontal base 1 as well as the corresponding locking hole 26 of the vertical support 2. One skilled in the art will readily appreciate that the shape of the locking holes is preferably configured to optimally receive locking mechanism. A locking hole is simply any hole or opening which receives the locking mechanism 22.

Alternatively, the locking mechanism 22 may mate with multiple corresponding locking holes (such as locking holes 27 and 28 as shown in FIGS. 10, 11, 13 and 21) of the horizontal base 1 as well as the corresponding locking hole 26 of the vertical support 2. In this embodiment, the locking hole 27 may be of a width that corresponds to the width of outer portion 23 while the locking holes 28 and 26 may be of a narrower width that corresponds to the narrower width of inner portion 24. One of ordinary skill in the art will readily appreciate that other locking mechanism configurations/shapes (and corresponding locking holes) are also available, and all such configurations/hole arrangements are intended to fall within the scope of this invention.

Additionally, the locking mechanism 22 may include recesses 25 along the outer portion 23 by which the band 17 such as an o-ring 6 may fit in (or be received in) to further hold in place the locking mechanism 22 (which further holds in place the vertical support 2 and the horizontal base 1).

Fastener or Attachment Mechanism

The fastener or attachment mechanism 39 for securing the patient's foot to the orthopedic device of the present invention include any fasteners or attachment mechanism or means known in the art, such as strap and buckle combination, ropes, VELCRO®, hook and loop fastener, touch fastener, ropes, buttons, etc., to be configured as needed to most comfortably or securely keep the orthopedic device on the patient's foot. For example, as shown in FIGS. 1-9 and 11, there may be a pair of straps 14 connected to each side of the horizontal base 1 as well as a pair of straps 14 connected to each side of vertical support 2. The straps 14 can be connected to the vertical support 2 through an opening 37 (e.g., a vertical slit) therein and can also be connected to horizontal support 1 through an opening 38 (e.g., a horizontal slit) therein.

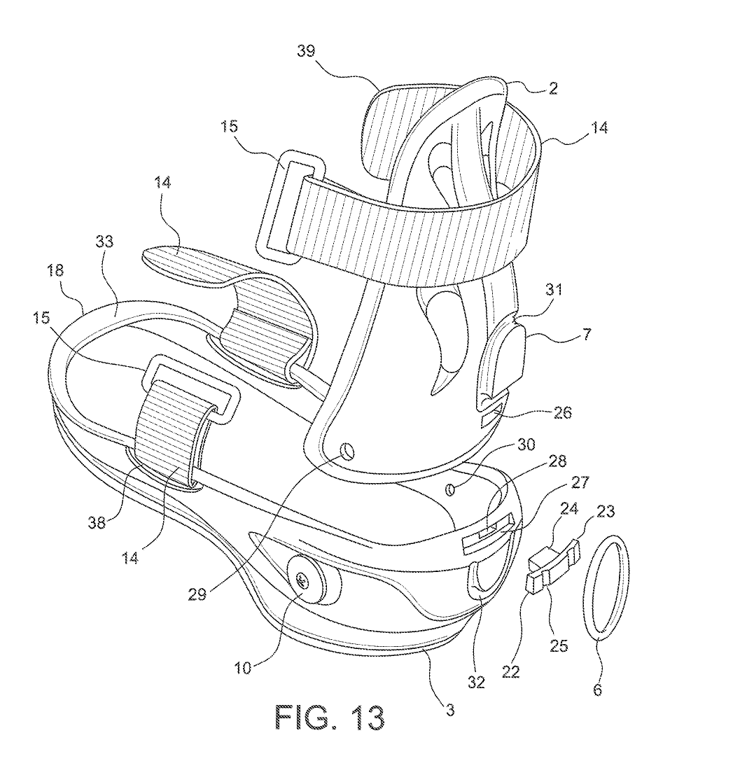

Alternatively, as shown in FIG. 13-16, there may be a strap 14 connected to each side of the horizontal base 1, and a single strap 14 and buckle 15 combination that is used to secure vertical support 2 to the patient's lower e.g., but is not connected to the vertical support 2 itself. Or, in yet another alternative embodiment as shown in FIGS. 17-19 and 21-24, the strap 14 can be connected to the vertical support 2 through opening 37 (e.g., a vertical slit) therein. In these exemplary embodiments, only two buckles 15 (as opposed to four shown in FIG. 1-9) are needed (e.g., one for the straps 14 connected to horizontal base 1 and one for the strap 14 securing vertical support 2). One skilled in the art will recognize that many other combination of straps and buckles (or any other fastener and attachment mechanism) can be used, and all are within the scope and spirit of this invention.

As shown in FIGS. 12, 14, 20 and 22, there may also be provided a liner 40 (e.g., a tongue) to shield the patient's exposed foot and/or lower leg area. The liner 40 may be a single piece that extend along the length of horizontal base 1 and along the height of vertical support 2, and may be made of foam or any other soft material known in the art. The liner 40 can also be of different lengths (e.g., compare the height of the liner or tongue 40 in FIG. 12 versus FIG. 20) as needed. The liner or tongue 40 may also include an additional shields or protectors 34 and. 35 on top of the liner 40 to further shield the patient's leg and foot from harm. The shields or protectors 34 and 35 may be attached to or a separate piece from liner 40, and can be made of a harder material such as plastic, rubber, metal or any other material known in the art. The liner 40 and the protectors 34 and 35 are secured to the patient's foot and lower leg area by fasteners such as straps 14 and buckles 15.

There is also provided herein a method for treating foot surgery with the orthopedic device of the present invention. In lieu of a conventional orthopedic device which does not move/pivot backwards and forward with the natural movement of the lower leg 13, the patient can instead wear the orthopedic device of the present invention after foot surgery as he/she recovers.

Having thus described the basic concept of the invention, it will be rather apparent to those skilled in the art that the foregoing detailed disclosure is intended to be presented by way of example only, and is not limiting. Various alterations, improvements, combinations and modifications will occur and are intended to those skilled in the art, though not expressly stated herein. These alterations, improvements, combinations and modifications are intended to be suggested hereby, and are within the spirit and scope of the invention.

1. An orthopedic device comprising:
a horizontal base having a front end and a rear end, and
a vertical support,
said vertical support being connected toward the rear end of said horizontal base by a pivot mechanism and a tension mechanism,
wherein said tension mechanism includes a band.

2. The orthopedic device of claim 1, wherein the band is an o-ring.

3. The orthopedic device of claim 1, wherein said vertical support includes a first protrusion for receiving said band, and
said horizontal base includes a second protrusion for receiving said band.

4. The orthopedic device of claim 3, wherein said horizontal base includes a first depression for receiving said band,
and said vertical support includes a second depression for receiving said band.

5. The orthopedic device of claim 1, wherein said band includes a tension indicator.

6. The orthopedic device of claim 1, further comprising a locking mechanism connecting said rear end of said horizontal base to said vertical support.

7. The orthopedic device of claim 6, wherein said locking mechanism includes one or more recesses for receiving said band.

8. The orthopedic device of claim 6, wherein said locking mechanism includes an outer portion having a first width and an inner portion having a second width.

9. The orthopedic device of claim 8, wherein said second width is narrower than said first width.

10. The orthopedic device of claim 1, further comprising an attachment mechanism.

11. The orthopedic device of claim 10, wherein said attachment mechanism includes a strap and buckle combination connected to said horizontal base and a strap and buckle combination connected to said vertical support.

12. The orthopedic device of claim 10, wherein said attachment mechanism includes a strap and buckle combination connected to said horizontal base and a strap and buckle combination that is not connected to said vertical support.

13. The orthopedic device of claim 10, further comprising a liner.

14. The orthopedic device of claim 13, further comprising a protector on top of said liner.

15. The orthopedic device of claim 1, wherein said horizontal base includes a lip to cover a patient's toes.

16. The orthopedic device of claim 1, further comprising
a locking mechanism connecting said rear end of said horizontal base to said vertical support,
a first attachment mechanism connected to said horizontal base and
a second attachment mechanism connected to said vertical support,
wherein said horizontal base includes a first protrusion for receiving said band and a first locking hole for mating with said locking mechanism, and
wherein said vertical support includes a second protrusion for receiving said band and a second locking hole for mating with said locking mechanism.

17. The orthopedic device of claim 16, wherein said locking mechanism includes one or more recesses for receiving said band.

18. The orthopedic device of claim 16, wherein said horizontal base includes a first depression for receiving said band,
and said vertical support includes a second depression for receiving said band.

19. A method for treating foot surgeries with the orthopedic device of claim 1.

20. A method for treating leg surgeries with the orthopedic device of claim 1.
